(19) United States
(12) Patent Application Publication
(10) Pub. No.: US 2026/0088135 A1
(43) Pub. Date: Mar. 26, 2026

(54) System and Method for Monitoring Exothermal Reactions in a Reactor

(71) Applicant: Siemens Aktiengesellschaft, Müenchen (DE)

(72) Inventors: Johannes BIERNATH, Karlsruhe (DE); Volker HIRSCH, Steinfeld (DE); Jürgen SCHMIDT, Neustadt an der Weinstraße (DE)

(21) Appl. No.: 19/111,349

(22) PCT Filed: Sep. 6, 2023
(86) PCT No.: PCT/EP2023/074359

§ 371 Date: Mar. 13, 2025

(30) Foreign Application Priority Data
Sep. 14, 2022 (DE) 102022209657.6

(51) Int. Cl. G16C 20/10; B01J 19/00; G16C 20/70

(52) U.S. Cl. CPC G16C 20/10; B01J 19/002; G16C 20/70; B01J 2219/00072

(57) ABSTRACT

A project planning system for creating a program for monitoring exothermal reactions in a reactor, wherein in order to create the program the project planning system provides at least one first functional module with a mathematical module for determining a maximum temperature and/or a maximum pressure in the reactor in the case of a continuous reaction, based on measured values and based on material data of components in the reactor, preferably by determining concentrations of components in the reactor, and at least one second functional module for determining the material data, in particular a heat capacity, density, vapor pressure, conductivity, solubility and/or viscosity of one or more components in the reactor, where the program is suitable, in particular, for implementation in a safety-oriented, memory-programmable controller.

### CROSS-REFERENCE TO RELATED APPLICATIONS

This is a U.S. national stage of application No. PCT/EP2023/074359 filed 6 Sep. 2023. Priority is claimed on German Application No. 10 2022 209 657.6 filed 14 Sep. 2022, the content of which is incorporated herein by reference in its entirety.

### BACKGROUND OF THE INVENTION

### 1. Field of the Invention

The invention relates to a computer program, a project engineering system and a method for creating a program for monitoring exothermic reactions in a reactor.

### 2. Description of the Related Art

Thermal runaway is the loss of temperature control of a chemical reaction due to an exothermic reaction, which can lead to an explosive temperature increase in a reactor. The loss of temperature control occurs when the generated heat, such as the heat of reaction, can no longer be sufficiently dissipated. The consequences of this are an increase in the speed of reaction and a process that is therefore self-accelerating, which releases further energy from the reaction or degradation process.

As a result, the temperature in the reactor increases and thus, as a rule, so too does the pressure. This is also referred to as a runaway reaction. This runaway reaction or the self-acceleration leads to the activation of safety facilities, for example, safety valves or rupture disks, and causes an escape of product (or, in the worst case, an explosion of the reactor), during the sudden release of which it is possible for high gas emissions to occur, which possibly may even be flammable or toxic.

In particular, this problem occurs in reactors with exothermic reactions that are operated in a semibatch operating mode. In this context, inter alia, the reaction may die out and there may be the simultaneous concentration of an added reagent, which is known as accumulation. When the reaction is reinstated, this accumulation can lead to an exponential release of energy, which can only be controlled with difficulty due to the self-accelerating reaction.

For this reason, it is desirable to already ascertain the hazard potential of a runaway reaction in advance via early detection methods.

The publication Biernath, Johannes, et al. “Model-based zero emission safety concept for reactors with exothermal reactions for chemical plants.” Journal of Loss Prevention in the Process Industries 72 (2021): 104494 gives an overview of conventional “online” model-based early detection methods. These methods use either a divergence criterion, an adiabatic criterion or an accumulation criterion.

The divergence method based on the divergence criterion, which is also the subject matter of EP 0 882 499 A1, describes how volume is generated via the temperature changes of the reactor, the speed of temperature change and the speed of temperature difference between reactor and cooling jacket. If there is an increase in the volume over time in this context, then it is apparent that a runaway reaction is present. In the publications, this method shows that the time between the detection and occurrence of a runaway reaction only amounts to a brief period of time, meaning that countermeasures are only possible to a limited extent, depending on the reaction acceleration.

For the accumulation criterion, an accumulation of a component added to the reaction (i.e., a reagent) is ascertained, and from this a maximum temperature and a maximum pressure in the reactor in the event of a runaway reaction are ascertained. To ascertain the accumulation, direct concentration measurements with the aid of optical methods or IR spectroscopy or indirect methods, such as an energy balance approach, for example, are cited in this context. The maximum temperature is compared with a design temperature of the reactor, for example, and if a threshold value is exceeded, for example, then the addition of the reaction component is stopped.

Further details regarding the accumulation criterion and regarding the calculation of an adiabatic temperature increase in the event of a runaway reaction are described in the publication Schmidt C., Biernath J., Schmidt J., Denecke J., 2022, Protection of chemical reactors against exothermal runaway reactions with smart overpressure protection devices, Chemical Engineering Transactions, 90, 493-498 DOI: 10.3303/CET2290083. Further details regarding the energy balance approach are also disclosed here.

For the further prior art, reference is made to WO 03/103826 A1 (in relation to the energy balance approach, inter alia) and to WO 00/47632 A1.

EP 3 621 726 B1 discloses a model-based method for monitoring the exothermic reactions in a semibatch reactor with the aid of the energy balance approach. In this context, an accumulation of at least one reaction component is ascertained and, on this basis, a maximum temperature in the reactor in the event of a runaway reaction is ascertained. This occurs with the aid of measurement values and ascertained substance data of components in the reactor.

One challenge in the early detection methods known from the prior art lies in the transition to a safety-related programmable logic controller (sPLC). These controllers limit the number of mathematical blocks, cyclical processing actions, provide only a limited programming language (LVL) and are also subject to standardization requirements in accordance with International Electrotechnical Commission (IEC) standard 61511, inter alia.

A programming of various control and monitoring programs by a linking of functional blocks contained in a library is known, for example, from WO 2021/076093 A1 and DE 10 2018 216456 A1.

### SUMMARY OF THE INVENTION

In view of the foregoing, it is therefore an object of the present invention to provide a project engineering system and a method for creating a program for monitoring exothermic reactions in a reactor, which makes it possible for the monitoring to be able to be implemented in a safety-related programmable logic controller.

This and other objects and advantages are achieved in accordance with the invention by a method, a computer program for monitoring exothermic reactions in a reactor and a project engineering system for creating a program for monitoring exothermic reactions in a reactor provides, where in order to create the program,
at least a first functional module with a mathematical model for ascertaining a maximum temperature and/or a maximum pressure in the reactor in the event of a runaway reaction based on measurement values and based on substance data of components in the reactor, preferably via an ascertaining of concentrations of components in the reactor, and
at least a second functional module for ascertaining the substance data, in particular a thermal capacity, a density, a vapor pressure, a conductivity, a solubility and/or a viscosity of one or more components in the reactor.

In the method in accordance with the invention for creating the program for monitoring exothermic reactions in a reactor, the program is created from
at least a first functional module with a mathematical model for ascertaining a maximum temperature and/or a maximum pressure in the reactor in the event of a runaway reaction based on measurement values and based on substance data of components in the reactor, preferably via an ascertaining of concentrations of components in the reactor, and
at least a second functional module for ascertaining the substance data, in particular a thermal capacity, a density, a vapor pressure, a conductivity, a solubility and/or a viscosity of one or more components in the reactor.

The computer program in accordance with the invention for monitoring exothermic reactions in a reactor comprises
at least a first functional module with a mathematical model for ascertaining a maximum temperature and/or a maximum pressure in the reactor in the event of a runaway reaction based on measurement values and based on substance data of components in the reactor, preferably via an ascertaining of concentrations of components in the reactor, and
at least a second functional module for ascertaining the substance data, in particular a thermal capacity, a density, a vapor pressure, a conductivity, a solubility and/or a viscosity of one or more components in the reactor.

Due to a modular construction of this kind, a flexible interconnection of modules of this kind and the parameterization thereof, it is possible to adjust to different processes, substances and installation sizes in a simple manner. The creation of the program can thus occur in a very efficient manner, and systematic errors can be avoided.

In the mathematical model, the ascertainment of the maximum temperature and/or the maximum pressure in the reactor in the event of a runaway reaction occurs based on measurement values and based on substance data of components in the reactor, preferably via an ascertainment of concentrations of components in the reactor. In other words, the ascertainment of the concentrations is likewise included by the mathematical model, and an ascertainment of concentrations of components in the reactor occurs first and then the ascertainment of the maximum temperature and/or the maximum pressure in the reactor occurs on this basis.

In this context, the second functional module can also contain pure substance equations; however, it is also possible for only a conversion of units to be used, for example.

In this context, the first functional module and/or the second functional module can also have installation variables as input variables (a reactor volume, for example).

For simple feeding and ascertaining of substance data, in this context at least the second functional module preferably comprises an interface to capture constants of pure substance equations, in particular from a substance database.

For simple programming, the functional modules are preferably provided as elements (for example, as blocks or what are known as typical modules) in a library.

In accordance with the invention, the program comprises commands which, when the program is executed by a computer, prompt it to perform a method in which, to monitor the exothermic reactions in the reactor, an accumulation of at least one reaction component in the reactor is ascertained, and based on this a maximum temperature and/or a maximum pressure in the reactor in the event of a runaway reaction is ascertained. In this context, the accumulation of the reaction component can be ascertained with the aid of measurement values, such as temperature, pressure and/or acoustic velocity in the reactor, for example. In this context, the monitoring preferably occurs “online” (i.e., in real time) based on “online” measurement values.

As has become apparent, it is possible to ascertain the accumulation of the reaction component and, based on this, a maximum temperature and/or a maximum pressure in a particularly simple manner, and thus also in a manner suitable for an implementation in a safety-related programmable logic controller, if the accumulation of the reaction component is ascertained with the aid of measurement values of an acoustic velocity in the reactor. This method can be subdivided very simply into various parts that build upon one another and can then be implemented as functional modules that are each separate. Only the measurement values, substance data and kinetic data of the production are necessary as input variables, for example. The substance data, for example, can be captured (automatically or via an operator) from a substance database, such as the VDI Heat Atlas. For the ascertainment of the maximum temperature and/or the maximum pressure, only relatively simple mathematical calculations are then necessary, which can also be implemented in a safety-related programmable logic controller with its limited mathematical possibilities and the cyclical program processing.

An acoustic velocity measurement for ascertaining gas fractions in a gas mixture is already known in principle from the publication Bates R., et al “Implementation of Ultrasonic Sensing for High Resolution Measurement of Binary Gas Mixture Fractions”, Sensors 2014, 14, 11260-11276; doi: 10.3390/s140611260. As has become apparent, this kind of measurement of the acoustic velocity particularly advantageously can also be used for a measurement of the molar concentrations in a reactor and, on this basis, a concentration of the reaction component and/or other components in the reactor and the accumulation thereof, for example, can be ascertained with relatively simple equations.

In principle, in this context it is possible to perform a measurement in a liquid or in a gas-filled space of the reactor.

Preferably, the acoustic velocity relates to a velocity of a sound in the ultrasonic range, i.e., sound waves above the limit of human hearing. As has become apparent, this sound range can be measured effectively and has few interfering influences.

In accordance with one advantageous embodiment, the ascertainment of the maximum temperature and/or the maximum pressure additionally occurs with the aid of measurement values of a density, a temperature and a pressure in the reactor (or of variables, from which it is possible to derive the aforementioned variables). This makes it possible to yet further simplify the mathematical calculations of the accumulation of the reaction component and thus the ascertaining of the maximum temperature and/or the maximum pressure.

In accordance with a further advantageous embodiment, a concentration of components in the reactor is ascertained with the aid of the measurement values.

In accordance with a further advantageous embodiment, an output signal for an activation of a safety response is generated when the ascertained maximum temperature and/or the maximum pressure exceeds a threshold value. The output signal, for example, can trigger an alarm for an operator of the reactor, who then triggers a safety response on their side, or alternatively can trigger an automatic safety response directly.

The threshold value is preferably derived from a design limit value of the reactor or an activation value of a safety facility of the reactor.

In the simplest case, the safety response comprises a reduction or a termination (stop) of a supply of the reaction component to the reactor and/or an increase in a cooling of the reactor.

As the danger of runaway reactions is particularly high in a semibatch operating mode, it is greatly advantageously used when the reactor is operated in a semibatch operating mode.

In accordance with a particularly advantageous embodiment, the program is a fail-safe program of a safety-related controller, in particular a safety-related programmable logic controller.

The project engineering system, project engineering method and computer program described above can also be used in cases in which an accumulation of a reaction component is also, for example, ascertained without measurement values of an acoustic velocity, such as with the aid of optical methods and/or an energy balance calculation. They are particularly suitable for creating fail-safe programs, in particular for a safety-related programmable logic controller.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

The invention as well as further advantageous embodiments of the invention in accordance with features of the subclaims are explained in greater detail below with reference to exemplary embodiments in the figures, but are not restricted to these in which:

FIG. 1 shows a schematic representation of a reactor with a controller for monitoring an exothermic reaction in the reactor in accordance with the invention;

FIG. 2 shows an exemplary creation of a program for monitoring an exothermic reaction in the reactor in accordance with the invention; and

FIG. 3 is a flowchart of the method in accordance with the invention.

### DETAILED DESCRIPTION OF THE EXEMPLARY EMBODIMENTS

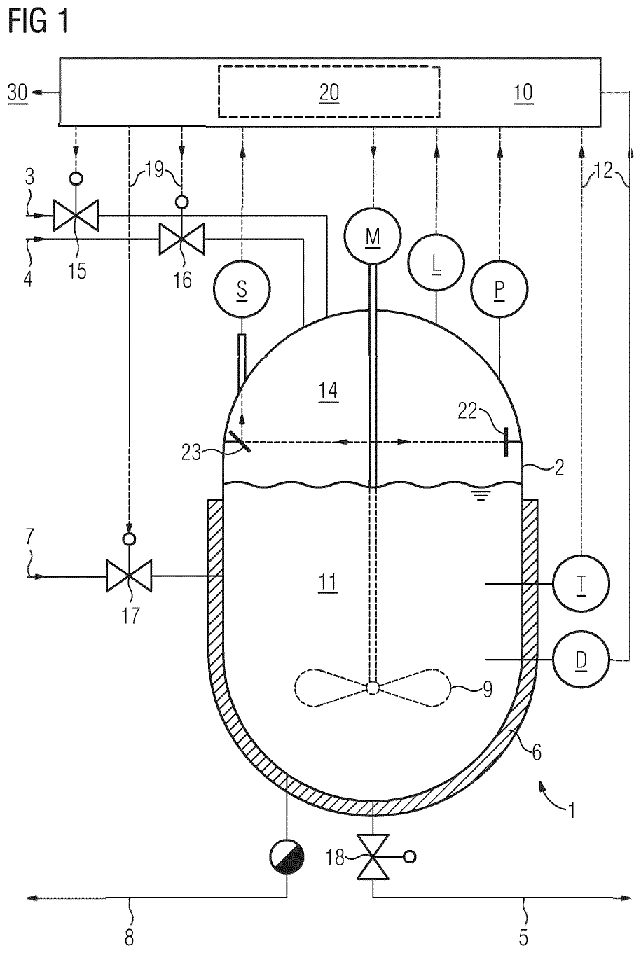

FIG. 1 shows a simplified schematic representation of a chemical reactor 1 with a reactor vessel 2, a plurality of supply lines to the vessel 2 for reactants and further auxiliary substances (two supply lines 3, 4 are shown in FIG. 1 by way of example), a discharge line 5 from the vessel 2 for a reaction product, and a cooling jacket 6, where it is possible for a coolant to be supplied thereto via a supply line 7 and discharged therefrom via a discharge line 8. Arranged in the vessel 2 is a stirrer 9, the shaft of which is guided upward out from the vessel and is driven by a motor M. In the vessel 2, a liquid phase mixture 11 is located below and a gas-filled space 14 is located above.

With the aid of various measurement value recorders (sensors), it is possible to measure physical variables in the reactor and in the supply and discharge lines. A pressure sensor P for measuring the pressure in the reactor vessel 2, a temperature sensor T for measuring the temperature of the liquid phase mixture 11 in the reactor vessel 2, a densimeter D for measuring an average density of the liquid phase mixture 11, a fill level measuring device L for measuring a fill level of the liquid phase mixture 11 and an acoustic velocity measuring device S for measuring an acoustic velocity in the gas-filled space 14 are examples in FIG. 1. Yet further measurement value recorders could be present, such as flow meters in the supply lines and discharge lines, and/or temperature sensors in the cooling jacket 6.

A controller 10 is used to control and monitor the reactor 1. Preferably, this involves a safety-related, fail-safe controller such as a fail-safe SIMATIC S7 from the applicant, for example. The controller captures the measurement values generated by the measurement value recorders (sensors), and to this end is connected to the measurement value recorders P, T, D, L, S via signal lines 12, which are shown schematically. In turn, the controller 10 can control a supply or discharge of reactants, auxiliary substances, reaction products and coolant via actuators, such as valves in the supply and discharge lines (see valves 15, 16, 17, 18), and to this end is connected to the actuators via control lines 19.

An exothermic reaction of reactants occurs in the reactor 1. In this context, the reactor is operated in a semibatch operating mode, for example. Due to a fail-safe programming 20 in the controller 10, a model-based continuous online method for monitoring the exothermic reaction in the reactor is now performed, i.e., the monitoring occurs in real time.

In this method, an accumulation of at least one reaction component (usually a reagent) in the reactor is ascertained and, based on this, a maximum temperature and/or a maximum pressure in the reactor in the event of a runaway reaction is ascertained. In this context, the accumulation of the reaction component is ascertained by the fail-safe programming 20 with the aid of the measurement values, described above, of the acoustic velocity, the temperature, the density and the pressure in the reactor 1.

The measurement of the acoustic velocity by the acoustic velocity measuring device S preferably occurs in the ultrasonic range. For the acoustic velocity measuring device S, it is possible to use an ultrasonic sensor of the type Echomax xps-10 in conjunction with a SITRANS LUT400 ultrasonic evaluation device from the applicant, for example. The acoustic velocity measuring device S or the associated sound sensors sit above on the vessel 2, for example, and thus feed the sound into the vessel in a vertical direction from above or receive sound above that has been reflected from a reflector element 22. The reflector element 22 is arranged in the gas-filled space 14 in the vessel 2. In principle, however, it is also possible for the sound to be fed in or received in the horizontal direction.

In order to extend the distance traveled and thus increase the accuracy of the measurement, the sound is diverted via a diverting element 23, which is likewise arranged in the gas-filled space 14 in the vessel 2, into a direction perpendicular to the feed-in direction (i.e., into a horizontal direction). The sound diverted in this manner is then reflected by the reflector element 22 and delivered back to the acoustic velocity measuring device S via the same path—only in the reverse direction (i.e., in turn via the diverting element 23).

A threshold value for a maximum temperature and/or a maximum pressure is stored in the controller 10. The threshold value is derived, for example, from a design limit value of the reactor or an activation value of a safety facility of the reactor. The controller 10 or the fail-safe program 20 now continuously compares the ascertained maximum temperature and/or the maximum pressure and, if these are exceeded, generates either an output signal 30 (for example, an alarm) for an operator, who can then trigger a safety response, or automatically triggers a safety response itself. A possible safety response could be, for example, an increase in the supply of coolant to the cooling jacket or a reduction or termination of a supply of the reagent to the reactor 1.

The exemplary embodiment of an exemplary chemical reaction, which should not merely be restricted thereto, is explained below. In this context, this involves an esterification of acetic anhydride with methanol to generate acetic acid and methyl acetate (i.e., four components):

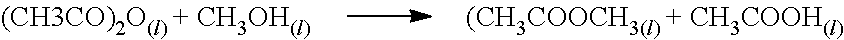

### Abbreviations

$$A = acetic anhydride$$

$$B = methanol$$

$$C = methyl acetate ( byproduct )$$

$$D = acetic acid ( main product )$$

1) Measurement of the average liquid density and mixture via the reciprocal approach of the pure substance densities weighted by the mass fraction (for example, according to VDI WA):

$$\text{?}$$

$$\text{?} \text{indicates text missing or illegible when filed}$$

ρₘ: average density of the liquid phase (measured with densimeter D)
ρᵢ: density of the component i (i=A, B, C, D)
xᵢ: mole fraction of the component i in the liquid phase (i=A, B, C, D)
Mᵢ: molar mass of the component i in the liquid phase (i=A, B, C, D)
2) Measurement of pressure in the gas-filled space 14 using the pressure sensor P (composed of nitrogen blanketing and partial pressures of the components):

$$\text{?}$$

$$\text{?} \text{indicates text missing or illegible when filed}$$

p: pressure in the gas-filled space 14 (measured)
p^{s}ᵢ: partial saturation pressure of the component i (i=A, B, C, D)
n_{N2}: amount of substance, nitrogen
R: molar gas constant
T: absolute temperature
V_{g}: volume of the gas-filled space
3) Measurement of acoustic velocity in the gas-filled space 14:

$$\text{?}$$

$$\text{?} \text{indicates text missing or illegible when filed}$$

c_{p,g,i}: specific thermal capacity of the component i (i=B, C, N2) with constant pressure
c_{p,v,i}: specific thermal capacity of the component i (i=B, C, N2) with constant volume
p: pressure in the gas-filled space 14
Mᵢ: molar mass of the component i (i=B, C, N2)
pc^{s}ᵢ: critical saturated vapor pressure of the component i
Note: In the above formula for measuring sound, components that form comparatively little vapor (here components A and D) are not taken into consideration, in order to simplify the calculation.
4) Final condition of dividing the components in the liquid phase:

$$1 = {x}_{A} + {x}_{B} + {x}_{C} + {x}_{D}$$

Thus, there are four unknown mole fractions (or concentrations) and four equations to this end, i.e., this system can be solved algebraically.

The further steps are:
a) Resolving the equation system in accordance with the accumulated amount x_{B} of the added amount B.
b) Determining the accumulated molar concentration CB of the added amount B from the accumulated amount x_{B} in a known manner via a conversion of the amount of substance.
c) Determining the adiabatic temperature increase via the accumulated mass

$$Δ {T}_{ad} = {c}_{B} \frac{Δ {H}_{R}}{\overline{ρ} {\overline{c}}_{p}}$$

$${c}_{B} = accumulated molar concentration of the added amount B$$

p̅: average density of the liquid phase
c̅ₚ: average specific thermal capacity across all components of the liquid phase
ΔHR: reaction enthalpy
ΔT_{ad}: adiabatic temperature increase
d) Determining the adiabatic pressure increase Δp_{ad} from the adiabatic temperature increase ΔT_{ad} with the aid of the general gas equation.

Starting from the current temperature or the current pressure in the reactor, it is possible to use the adiabatic temperature increase or the adiabatic pressure increase to ascertain a maximum temperature or a maximum pressure in the reactor in the event of a runaway reaction.

In the example in FIG. 1, four substance components were used or equations for four substance components were formulated. In a similar manner, it should be understood it is also possible to use fewer or more substance components or to formulate corresponding equations for a greater or smaller number of components.

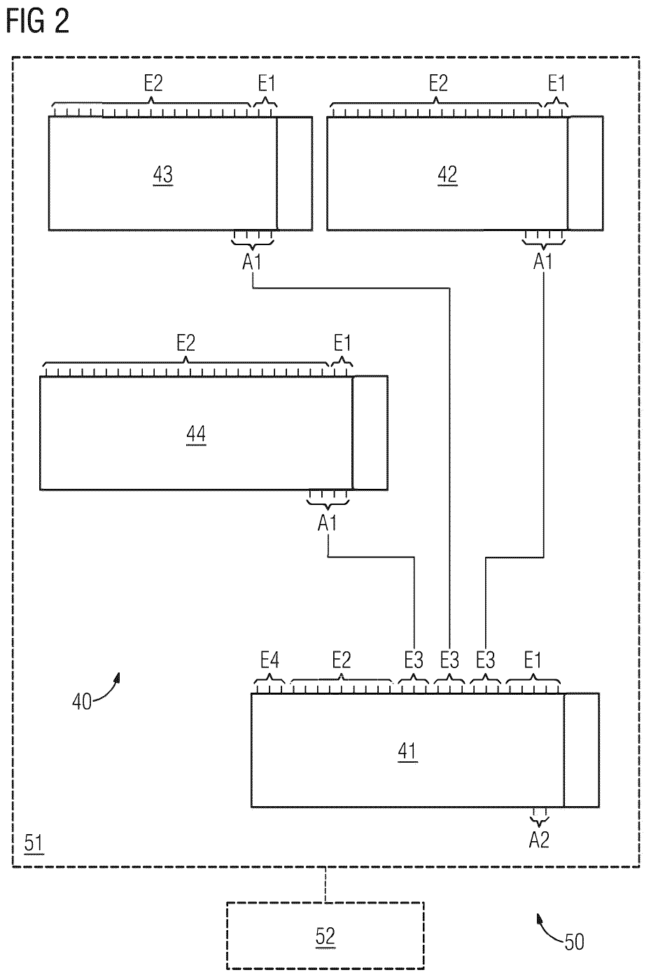

FIG. 2 shows, by way of example, a creation of a program for monitoring exothermic reactions in a reactor, in particular for the fail-safe program 20 in the controller 10 from FIG. 1.

In this context, FIG. 2 shows a view that is offered to a project engineer for creating the program on a user interface 51 of a project engineering system 50. The project engineering system 50 furthermore comprises a central computing unit 52, such as a PC.

The fail-safe programming comprises a plurality of functional modules 41, 42, 43, 44.

A first functional module 41 comprises a mathematical model, such as the model described above by way of example, for ascertaining the maximum temperature and/or the maximum pressure in the event of a runaway reaction based on measurement values and based on substance data of the components in the reactor. Depending on requirements, it is possible to add yet further functional models for ascertaining the maximum temperature and/or the maximum pressure in the event of a runaway reaction based on measurement values and based on substance data of the components in the reactor.

The functional modules 42, 43, 44 are used to ascertain and provide the substance data (and further kinetic data, as appropriate) of one or more components in the reactor for the functional module 41; the functional module 42 is used to ascertain vapor pressures, the functional module 43 is used to ascertain densities and the functional module 44 is used to ascertain thermal capacities. Depending on requirements, it is possible to add yet further functional models for ascertaining and providing the substance data (and further kinetic data, as appropriate) of one or more components in the reactor.

Pure substance equations are stored in each of the functional modules 41, 42, 43, 44 for the ascertaining of substance data (and further kinetic data, as appropriate).

Each of the functional modules 41, 42, 43, 44 has inputs E1 for measurement values and inputs E2 for constants of the pure substance equations. Inputs E4 for installation variables (for example, a reactor volume) can also be present.

The constants of the pure substance equations can be captured, for example, by an operator or on an automated basis from a pure substance database, such as the VDI Heat Atlas.

Each of the functional modules 42, 43, 44 has outputs A1 for the ascertained substance data, which in turn are connected to corresponding inputs E3 of the functional module 41. The functional module 41 has outputs A2, which for example already output information regarding an exceeding of a threshold value, i.e., an imminent hazardous situation. However, it is also possible for only an ascertained maximum temperature and/or maximum pressure to be output via the outputs A2, for example, and the comparison with a threshold value occurs outside of the functional block 41.

In this context, the outputs A1, A2 can also comprise a BAD signal, which signals a faulty calculation. For example, the functional modules 42, 43, 44 are each linked to the functional module 41 via a BAD signal, meaning that in the event of faulty calculations the functional module 41 also has a BAD signal at output A2, which can be output to an operator as an alarm.

Preferably, the functional modules are provided as elements (known as typical modules) in a block library for the fail-safe programming of the controller 10.

Due to the modular structure described, a project engineer can select the modules required from the library in a flexible manner for their respective application case, interconnect them and parameterize them. The creation of the program can therefore occur in a very efficient manner and free from systematic errors.

The program can be used in a safety-related controller, but does not have to be. For example, the program can also be used as a monitoring function in a non-safety-related application (for example, based in the Cloud). This application can be used to monitor a product quality, for example.

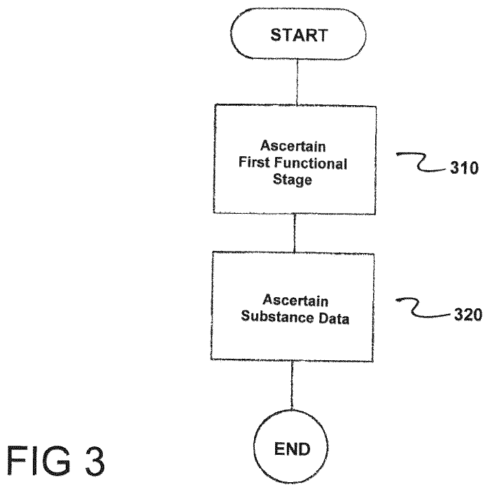

FIG. 3 is a flowchart of the method for creating a program for monitoring exothermic reactions in a reactor. In order to create the program, the method comprises ascertaining, via at least a first functional stage including a mathematical model, at least one of a maximum temperature and a maximum pressure in the reactor in an event of a runaway reaction based on measurement values of physical variables in the reactor and based on substance data of components in the reactor via an ascertainment of concentrations of components in the reactor, as indicated in step 310.

Next, the substance data is ascertained via a second functional stage, as indicated in step 320. Here, the substance data comprising at least one of a thermal capacity, a density, a vapor pressure, a conductivity, a solubility and a viscosity of at least one component in the reactor.

In accordance with the method of the invention, at least the second functional module comprises an interface to capture constants of pure substance equations from a substance database.

In addition, the program comprises commands which, when the program is executed by a computer, prompt it to perform a method in which, to monitor the exothermic reactions in the reactor, an accumulation of at least one reaction component in the reactor is ascertained, and based on this a maximum temperature and/or a maximum pressure in the reactor in the event of a runaway reaction is ascertained.

Thus, while there have been shown, described and pointed out fundamental novel features of the invention as applied to a preferred embodiment thereof, it will be understood that various omissions and substitutions and changes in the form and details of the methods described and the devices illustrated, and in their operation, may be made by those skilled in the art without departing from the spirit of the invention. For example, it is expressly intended that all combinations of those elements and/or method steps that perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1.-15. (canceled)

16. A project engineering system for creating a program for monitoring exothermic reactions in a reactor, in which in order to create the program, the project engineering system comprising:
at least a first functional stage including a mathematical model for ascertaining at least one of a maximum temperature and a maximum pressure in the reactor in an event of a runaway reaction based on measurement values of physical variables in the reactor and based on substance data of components in the reactor via an ascertainment of concentrations of components in the reactor; and
at least a second functional stage for ascertaining the substance data, the substance data comprising at least one of a thermal capacity, a density, a vapor pressure, a conductivity, a solubility and a viscosity of at least one component in the reactor;
wherein at least the second functional module comprises an interface to capture constants of pure substance equations, in particular from a substance database; and
wherein the program comprises commands which, when executed by a processor of a computer, cause the exothermic reactions in the reactor to be monitored such that an accumulation of at least one reaction component in the reactor is ascertained, and based on this at least one of a maximum temperature and a maximum pressure in the reactor in an event of a runaway reaction is ascertained.

17. The project engineering system as claimed in claim 16, wherein the program is a fail-safe program of a safety-related controller, in particular a safety-related programmable logic controller.

18. The project engineering system as claimed in claim 17, wherein the safety-related controller is a safety-related programmable logic controller.

19. The project engineering system as claimed in claim 16, wherein the functional modules are provided as elements in a library for fail-safe programming of a controller.

20. The project engineering system as claimed in claim 17, wherein the functional modules are provided as elements in a library for fail-safe programming of a controller.

21. A method for creating a program for monitoring exothermic reactions in a reactor, the method comprising:
ascertaining, via at least a first functional stage including a mathematical model, at least one of a maximum temperature and a maximum pressure in the reactor in an event of a runaway reaction based on measurement values of physical variables in the reactor and based on substance data of components in the reactor via an ascertainment of concentrations of components in the reactor; and
ascertaining, via a second functional stage, the substance data, the substance data comprising at least one of a thermal capacity, a density, a vapor pressure, a conductivity, a solubility and a viscosity of at least one component in the reactor;
wherein at least the second functional module comprises an interface to capture constants of pure substance equations from a substance database; and
wherein the program comprises commands which, when the program is executed by a computer, prompt it to carry out a method in which, to monitor the exothermic reactions in the reactor, an accumulation of at least one reaction component in the reactor is ascertained, and based on this a maximum temperature and/or a maximum pressure in the reactor in the event of a runaway reaction is ascertained.

22. The method as claimed in claim 21, wherein the program is a fail-safe program of a safety-related controller.

23. The method as claimed in claim 22, wherein the safety-related controller is a safety-related programmable logic controller.

24. The method as claimed in claim 21, wherein the functional modules are provided as elements in a library for fail-safe programming of a controller.

25. The method as claimed in claim 22, wherein the functional modules are provided as elements in a library for fail-safe programming of a controller.

26. A non-transitory computer-readable medium encoded with a computer program comprising commands which, when executed by a processor of a computer, exothermic reactions in a reactor to be monitored such that an accumulation of at least one reaction component in the reactor is ascertained, and based on this at least one of a maximum temperature and a maximum pressure in the reactor in the event of a runaway reaction is ascertained, the computer program comprising:
at least a first functional module with a mathematical model for ascertaining at least one of a maximum temperature and a maximum pressure in the reactor in an event of a runaway reaction based on measurement values of physical variables in the reactor and based on substance data of components in the reactor via an ascertainment of concentrations of components in the reactor; and
at least a second functional module for ascertaining the substance data, the substance data comprising at least one of a thermal capacity, a density, a vapor pressure, a conductivity, a solubility and a viscosity of at least one component in the reactor;
wherein at least the second functional module comprises an interface to capture constants of pure substance equations, in particular from a substance database; and

27. The non-transitory computer-readable medium claimed in claim 26, wherein the program is a fail-safe program of a safety-related controller.

28. The non-transitory computer-readable medium as claimed in claim 27, wherein the safety-related controller is a safety-related programmable logic controller.

29. The non-transitory computer-readable medium as claimed in claim 26, wherein the functional modules are provided as elements in a library for fail-safe programming of a controller.

30. The non-transitory computer-readable medium as claimed in claim 27, wherein the functional modules are provided as elements in a library for fail-safe programming of a controller.
